(19) United States
(12) Patent Application Publication
(10) Pub. No.: US 2006/0039812 A1
(43) Pub. Date: Feb. 23, 2006

(54) Pulsation damper designed to ensure alignment of diaphragm during assembling

(76) Inventor: Toshiaki Agui, Sashima-gun (JP)

Correspondence Address: CLARK & BRODY, 1090 VERMONT AVENUE, NW, SUITE 250, WASHINGTON, DC, 20005, US

(21) Appl. No.: 11/205,161

(22) Filed: Aug. 17, 2005

(30) Foreign Application Priority Data
Aug. 18, 2004 (JP) 2004-237846

(51) Int. Cl. A61M 1/00; F04B 11/00

(52) U.S. Cl. 417/540; 417/437

(57) ABSTRACT

A pulsation damper for use in an automotive fuel supply system is provided which consists of a casing, a diaphragm, and a cover. The cover is joined to the casing with the diaphragm nipped therebetween. The pulsation damper also includes a supporting member secured to a central portion of the diaphragm to support the diaphragm physically. The supporting member has a protrusion fitted in a recess formed in the casing to hold the supporting member from moving in an unwanted direction, thereby ensuring coaxial alignment of the diaphragm with the casing when the cover is joined to the casing. The protrusion may alternatively be formed on the casing, while the recess may be formed in the supporting member.

### CROSS REFERENCE TO RELATED DOCUMENT

The present application claims the benefit of Japanese Patent Application No. 2004-237846 filed on Aug. 18, 2004, the disclosure of which is incorporated herein by reference.

### BACKGROUND OF THE INVENTION

1 Technical Field of the Invention

The present invention relates generally to a pulsation damper which may be employed in attenuating pulsations of fuel flowing in a fuel supply system for automotive vehicles, and more particularly to an improved structure of such a pulsation damper designed to ensure coaxial alignment of a diaphragm with a casing during assembling of the pulsation damper.

2 Background Art

FIG. 5 shows an example of a conventional pulsation damper for use in a fuel supply system of automotive vehicles. For example, International Publication No. WO99/60264 teaches such a pulsation damper.

The pulsation damper 1 includes a casing 2, a cover 3, and a diaphragm 4. The diaphragm 4 is disposed inside an assembly of the casing 2 and the cover 3 to define an air chamber 5 and a fuel chamber 6. The diaphragm 4 is exposed to fuel entering the fuel chamber 6 and works to oscillate in response to pulsations of the fuel to absorb or attenuate them.

The casing 2 has a flange crimped inwardly to nip a flange of the cover 3 and the periphery of the diaphragm 4. The diaphragm 4 is usually made of a thin film and required to be installed coaxially with the casing 2 with high accuracy. To this end, the diaphragm 4 has a support 8 installed in the center thereof. When subjected to no pressure of the fuel within the fuel chamber 6, the support 8 is placed in abutment of a bottom wall 8a thereof with an inner bottom wall 2a of the casing 2. The support 8 is also kept in this state when the casing 2 is jointed to the cover 3.

The structure of the pulsation damper 1, however, has the problem in that the inner bottom wall 2a of the casing 2 is curved, thus resulting in physical instability of the diaphragm 4 when the casing 2 is joined to the cover 3, which gives rise to misalignment of the diaphragm 4 with the casing 2 or undesirable securement of the diaphragm 4 in an inclined position. The coaxial alignment of the diaphragm 4 with the casing 2 before the casing 2 is joined to the cover 3 may be achieved by fitting the periphery of the diaphragm 4 to the inner periphery of the flange of the casing 2, which, however, results in a difficulty in ensuring the coaxial alignment upon joining of the casing 2 and the cover 3 because the diaphragm 4 is lower in rigidity.

### SUMMARY OF THE INVENTION

It is therefore a principal object of the invention to avoid the disadvantages of the prior art.

It is another object of the invention to provide an improved structure of a pulsation damper which is designed to ensure coaxial alignment of a diaphragm with a casing during assembling of the pulsation damper.

According to one aspect of the invention, there is provided a pulsation damper which may be used in attenuate pulsations of fuel flowing through a fuel supply system of an automotive vehicle. The pulsation damper comprises: (a) a casing having a recess formed therein; (b) a diaphragm disposed in the casing, having an outer periphery and a central portion, the diaphragm working to absorb pulsations of fluid entering a fluid chamber formed in the casing; (c) a cover joined to the casing together with the outer periphery of the diaphragm; and (d) a supporting member secured to the central portion of the diaphragm to support the diaphragm physically. The supporting member has a protrusion fitted in the recess of the casing to hold the supporting member from moving in an unwanted direction, thereby ensuring coaxial alignment or concentricity of the diaphragm with the casing when the cover is joined to the casing, which facilitates ease of joining the cover and the casing.

According to another aspect of the invention, there is provided a pulsation damper which comprises: (a) a casing having a protrusion formed thereon; (b) a diaphragm disposed in the casing, having an outer periphery and a central portion, the diaphragm working to absorb pulsations of fluid entering a fluid chamber formed in the casing; (c) a cover joined to the casing together with the outer periphery of the diaphragm; and (d) a supporting member secured to the central portion of the diaphragm to support the diaphragm physically. The supporting member has a recess in which the protrusion of the casing is fitted to hold the supporting member from moving in an unwanted direction, thereby ensuring coaxial alignment or concentricity of the diaphragm with the casing when the cover is joined to the casing, which facilitates ease of joining the cover and the casing.

In the preferred mode of the invention, a periphery of the casing is crimped to achieve a joint of the casing with the cover. The use of the supporting member serves to facilitate ease of crimping of the casing, thus resulting in a decrease in production cost of the pulsation damper.

The pulsation damper is designed to be used in an automotive fuel system.

### BRIEF DESCRIPTION OF THE DRAWINGS

The present invention will be understood more fully from the detailed description given hereinbelow and from the accompanying drawings of the preferred embodiments of the invention, which, however, should not be taken to limit the invention to the specific embodiments but are for the purpose of explanation and understanding only.

In the drawings:

FIG. 1 is a longitudinal sectional view which shows the structure of a pulsation damper according to the first embodiment of the invention;

FIG. 2 is an exploded longitudinal sectional view which shows the pulsation damper of FIG. 1 before being assembled;

FIG. 3 is a longitudinal sectional view which illustrates the process of joining of a cover and a casing of the pulsation damper of FIG. 1;

FIG. 4 is a longitudinal sectional view which shows the structure of a pulsation damper according to the second embodiment of the invention; and

FIG. 5 is a longitudinal sectional view which shows the structure of a conventional pulsation damper.

### DESCRIPTION OF THE PREFERRED EMBODIMENTS

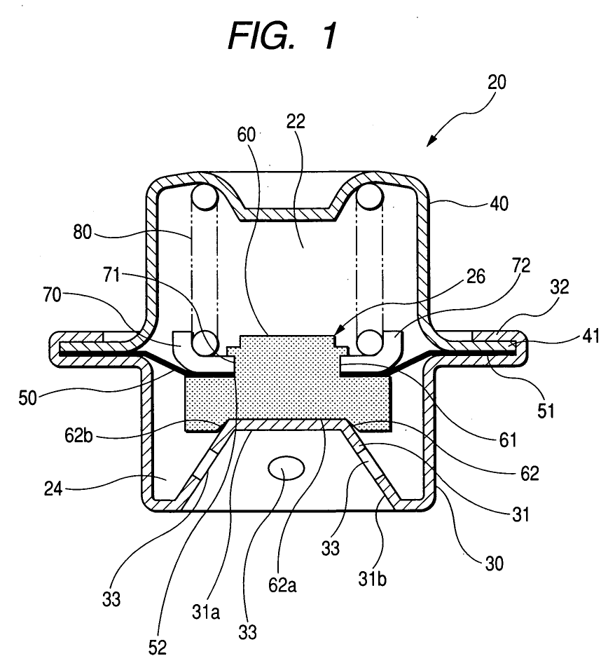

Referring now to the drawings, particularly to FIG. 1, there is shown a pulsation damper 20 according to the first embodiment of the invention. The pulsation damper 20 may be employed in various applications and will be discussed below in the case of use in a fuel system of automotive vehicles.

The pulsation damper 20 generally includes a casing 30, a cover 40, and a diaphragm 50. The casing 30 and the cover 40 are joined together to nip the diaphragm 50 therebetween. The diaphragm 50 defines an air chamber 22 and a fuel chamber 24 within an assembly of the casing and the cover 40. The diaphragm 50 is sensitive to vibrations or pulsations of fuel pressure within the fuel chamber 24 to attenuate or eliminate them.

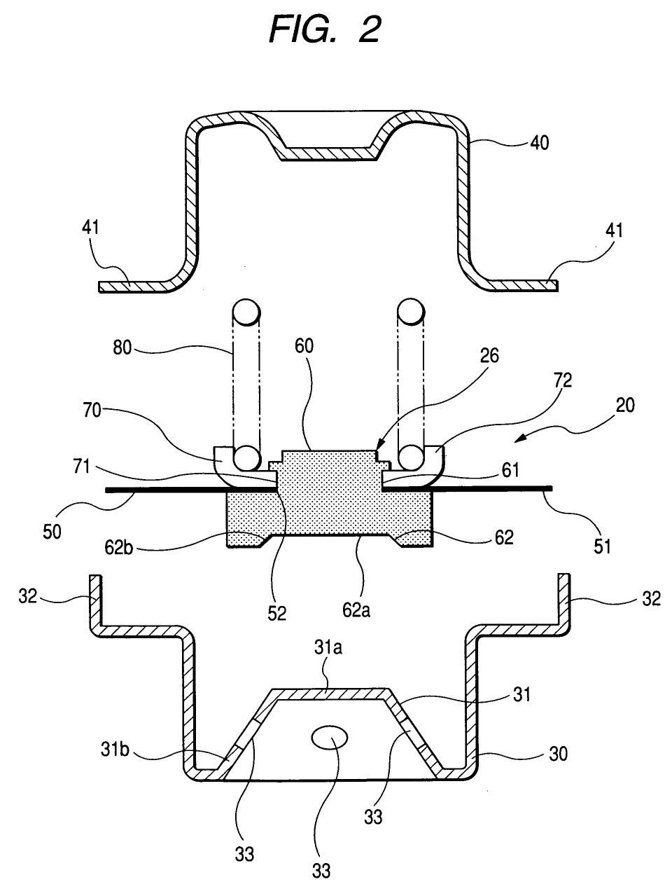

The casing 30 is of a hat-shape and has an annular flange 32 joined to the cover 40. Before joined to the cover 40, the flange 32, as illustrated in FIG. 2, is of an L-shape in cross section with an outer periphery extending vertically, as viewed in the drawing. The casing 30 also has a frusto-conical protrusion 31 on the center of an inner bottom wall thereof. The protrusion 31 is formed by a flat wall 31a and a tapered side wall 31b. The tapered side wall 31b has a plurality of openings 33 formed therein which work as fuel inlets.

The cover 40 is of a hat-shape and has an annular flange 41 extending horizontally. The flange 41 is, as clearly shown in FIG. 1, nipped firmly by inwardly bending or crimping the flange 32 of the casing 30 to retain the diaphragm 50.

The diaphragm 50 is made of a circular thin film and has an outer periphery affixed to the flange 41 of the cover 40 firmly by the flange 32 of the casing 30. The diaphragm 50 has a circular center opening 52 formed in the center thereof.

The pulsation damper 20 also includes a support reinforcement 60 which is fitted in the center opening 52 of the diaphragm 50 to retain and reinforce the diaphragm 50. The support reinforcement 60 is made up of two parts: an upper small-diameter portion and a lower large diameter portion. The upper small-diameter portion has an annular groove 61. The lower large-diameter portion has a frusto-conical recess 62 which is defined by a flat bottom 62a and a tapered side wall 62b and, as can be seen from FIG. 1, substantially contoured to conform with the contour of the protrusion 31 of the casing 30 so that the protrusion 31 is fitted in the recess 62 in abutment of the flat wall 31a thereof with the bottom 62a of the recess 62 to hold the support reinforcement 60 from moving laterally.

The pulsation damper 20 also includes a ring-shaped spring seat 70 and a coil spring 80. The spring seat 70 has a center opening 71 and a periphery 72 curved upwards. The center opening 71 has substantially the same diameter as that of the center opening 52 of the diaphragm 50. The spring 80 is disposed between the curved periphery 72 of the spring seat 70 and an inner wall of the cover 40. The spring seat 70 is fitted within the groove 61 of the support reinforcement 60 together with the diaphragm 50.

Specifically, the spring seat 70 is assembled with the diaphragm 50 and the support reinforcement 60 as a diaphragm unit 26 prior to the joining of the casing 30 and the cover 40. The assembling is achieved by fitting the periphery of the center opening 52 of the diaphragm 50 in the groove 61 of the support reinforcement 60, then fitting the periphery of the center opening 71 of the spring seat 70 in the groove 61, and finally staking the small-diameter portion of the support reinforcement 60 to retain the spring seat 70 and the diaphragm 50 firmly.

The assembling of the pulsation damper 20 will be described below. FIG. 2 illustrates the casing 30, the cover 40, the diaphragm unit 26, and the spring 80 before being assembled.

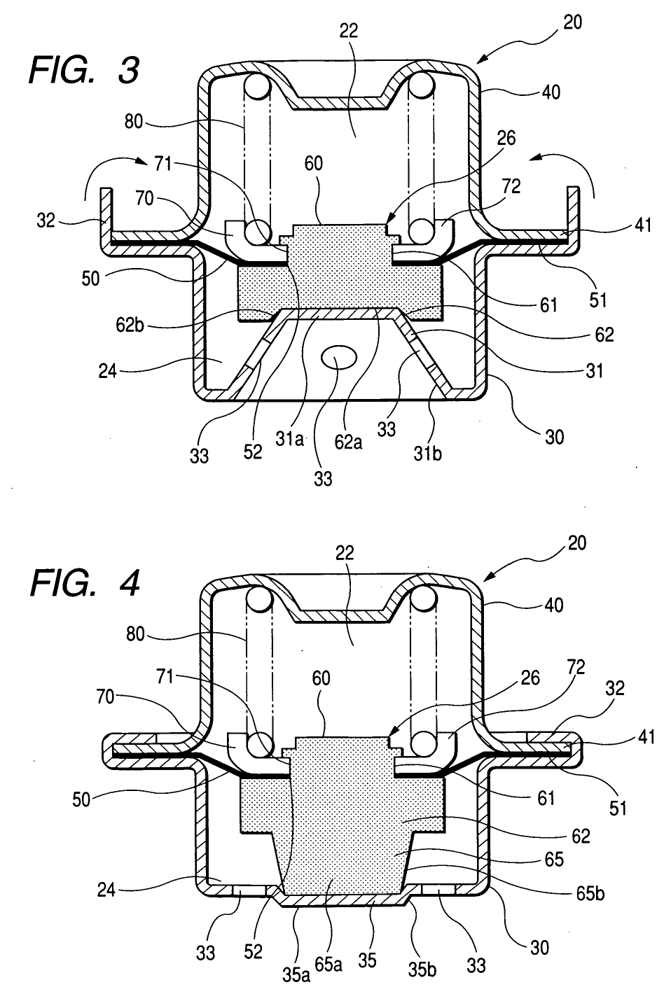

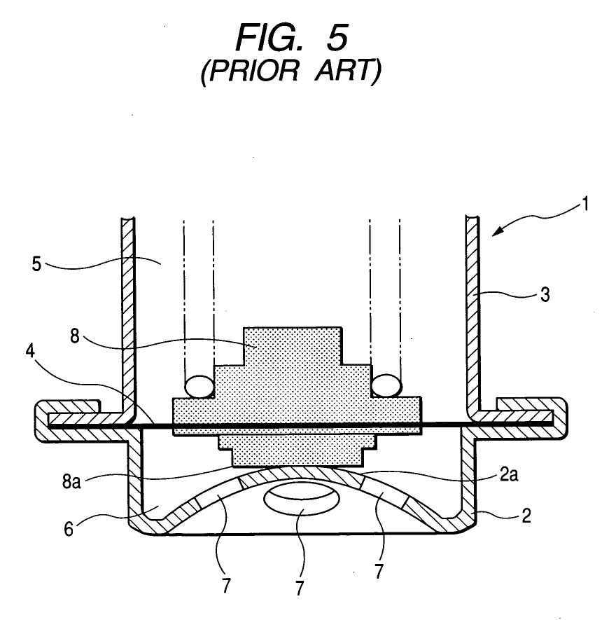

First, the diaphragm unit 26 is disposed in the casing 30 with the outer periphery 51 of the diaphragm 50, as illustrated in FIG. 3, placed on the flange 32 of the casing 30 inside the upright portion of the flange 32. The support reinforcement 60 is mounted on the protrusion 31 of the casing 30 with the recess 62 fitted on the protrusion 31. Specifically, the bottom 62a of the recess 62 is in direct contact with the flat wall 31a of the protrusion 31. Simultaneously, the tapered side wall 62b of the recess 62 is in direct contact with the tapered side wall 31b of the protrusion 31. This ensures the securement of the diaphragm unit 26 on the protrusion 31 of the casing 30.

Finally, the periphery or upright portion of the flange 32 of the casing 30 is bent inwardly, as indicated by arrows in FIG. 3, and elastically pressed or crimped to form a nip retaining the flange 41 of the cover 40 and the outer periphery 51 of the diaphragm 50 firmly. During this process, the diaphragm unit 26 is held from being moved laterally by the protrusion 31 of the casing 30 to ensure physical coincidence of the center of the diaphragm 50 with that of the casing 30.

In use, the pulsation damper 20 is installed in a fuel pipe of automotive vehicles. The fuel enters the fuel chamber 24 through the openings 33. If the fuel is pulsating, it will cause the diaphragm 50 to oscillate to smooth out the pulsation of the fuel.

FIG. 4 shows the pulsation damper 20 according to the second embodiment of the invention which is different from the one of the first embodiment in that a recess 35 is formed in an inner bottom of the casing 30, and a protrusion 65 is formed on the support reinforcement 60. Other arrangements are identical with those in the first embodiment, and explanation thereof in detail will be omitted here.

Specifically, the casing 30 is, like the first embodiment, of a hat-shape and has the annular flange 32, but has the recess 35 defined by a flat bottom 35a and a tapered side wall 35b. The openings 33 (i.e., the fuel inlets) are formed in the bottom of the casing 30 outside the recess 35.

The support reinforcement 60 has the frusto-conical protrusion 65 defined by a flat wall 65a and a tapered side wall 65b. The support reinforcement 60 is fitted within the recess 35 of the casing 30 in abutment of the flat wall 65a with the bottom 35a of the recess 35 and of the tapered side wall 65b with the tapered side wall 35b of the recess 35, thereby holding the support reinforcement 60 from moving laterally. Specifically, the diaphragm unit 26 is held firmly by the engagement of the protrusion 65 of the support reinforcement 60 with the recess 35 of the casing 30, thereby ensuring coaxial alignment of the diaphragm 50 with the casing 30 during the crimping of the flange 32 of the casing 30 to secure the diaphragm 50 within the pulsation damper 20 in a desired position.

While the present invention has been disclosed in terms of the preferred embodiments in order to facilitate better understanding thereof, it should be appreciated that the invention can be embodied in various ways without departing from the principle of the invention. Therefore, the invention should be understood to include all possible embodiments and modifications to the shown embodiments witch can be embodied without departing from the principle of the invention as set forth in the appended claims.

What is claimed is:

1. A pulsation damper comprising:
a casing having a recess formed therein;
a diaphragm disposed in said casing, having an outer periphery and a central portion, said diaphragm working to absorb pulsations of fluid entering a fluid chamber formed in said casing;
a cover joined to said casing together with the outer periphery of said diaphragm; and
a supporting member secured to the central portion of said diaphragm to support said diaphragm physically, said supporting member having a protrusion fitted in the recess of said casing to hold said supporting member from moving in an unwanted direction.

2. A pulsation damper as set forth in claim 1, wherein a periphery of said casing is crimped to achieve a joint of said casing with said cover.

3. A pulsation damper as set forth in claim 1, wherein the pulsation damper is designed to be used in an automotive fuel system.

4. A pulsation damper comprising:
a casing having a protrusion formed thereon;
a diaphragm disposed in said casing, having an outer periphery and a central portion, said diaphragm working to absorb pulsations of fluid entering a fluid chamber formed in said casing;
a cover joined to said casing together with the outer periphery of said diaphragm; and
a supporting member secured to the central portion of said diaphragm to support said diaphragm physically, said supporting member having a recess in which the protrusion of said casing is fitted to hold said supporting member from moving in an unwanted direction.

5. A pulsation damper as set forth in claim 4, wherein a periphery of said casing is crimped to achieve a joint of said casing with said cover.

6. A pulsation damper as set forth in claim 4, wherein the pulsation damper is designed to be used in an automotive fuel system.
